(19) United States
(12) Patent Application Publication
(10) Pub. No.: US 2026/0000908 A1
(43) Pub. Date: Jan. 1, 2026

(54) COMPACT THERAPEUTIC INFRARED DEVICE, CHARGING CASE AND CHARGING SYSTEM

(71) Applicant: MUNCHKIN, INC., Van Nuys, CA (US)

(72) Inventors: Fangyi GAO, Van Nuys, CA (US); Jaime SANCHEZ SOLANA, Van Nuys, CA (US); Agnes Yena LEE, Van Nuys, CA (US); Sung Yun CHAN, Van Nuys, CA (US); Robert Zacaries EVORA, Van Nuys, CA (US); Steven Bryan DUNN, Van Nuys, CA (US)

(21) Appl. No.: 19/255,994

(22) Filed: Jun. 30, 2025

Related U.S. Application Data
(60) Provisional application No. 63/665,710, filed on Jun. 28, 2024.

(51) Int. Cl. A61N 5/06; A61M 1/06

(52) U.S. Cl. CPC A61N 5/0616; A61M 1/064; A61N 2005/0632; A61N 2005/0645; A61N 2005/0651; A61N 2005/0659; A61N 2005/0668

(57) ABSTRACT

A compact IR therapeutic device having a cover, an arrangement of IR LED's, a flexible circuit board and a flexible overmold layer attached to the cover. The flexible circuit board is electrically connected to the IR LED's and provides various routine commands thereto. The flexible overmold layer secures the flexible circuit board to the cover. The IR LED's are secured to the flexible circuit board and disposed between the cover and the flexible overmold layer. The compact IR therapeutic device is adapted to be secured over a woman's nipple, and the IR LED's are illuminated to provide a therapeutic red light onto the woman's nipple for healing purposes.

### CROSS-REFERENCE TO RELATED APPLICATIONS

This application claims the benefit under 35 U.S.C. 119 of U.S. Provisional Patent Application No. 63/665,710, filed Jun. 28, 2024, and entitled “COMPACT THERAPEUTIC INFRARED DEVICE, CHARGING CASE AND CHARGING SYSTEM”, which application is incorporated by reference herein in its entirety.

### TECHNICAL FIELD

The subject disclosure relates generally to a therapeutic infrared device for healing skin. More particularly, the present disclosure relates to a compact infrared nipple coverage device and system to provide therapeutic healing.

### BACKGROUND

Infrared therapy involves the use of infrared radiation applied to skin in order to provide rejuvenating benefits, burn calories, release toxins, increase blood flow, case pain and/or influence a state of relaxation. Within the infrared spectrum, the most effective wavelength for providing therapeutic effects is in the red-light therapy wavelength range between 630 and 700 nanometers (nm).

Red light therapy uses low levels of red light to target and provide healing to a person's skin, cells, muscle tissue, and other parts of a body. Red light increases the production of mitochondria in cells' energy sources which improves cell function and repair. Red light therapy is sometimes referred to as low-level laser therapy, low-power laser therapy, low-power laser, or photobiomodulation. In red light therapy, an individual's skin is exposed to a lamp, device, or laser with a red light. It uses an LED (light-emitting diode) that emits the particular spectrum of light at a given wavelength. Red LED light travels deeper into the skin, which is used to treat various skin surface conditions. When the light enters your skin, your mitochondria soak it up and make more energy, which helps cells repair themselves.

Although these benefits are known, there is a need for a compact device capable of providing therapeutic healing effects in a discreet and inconspicuous manner to sensitive areas.

### SUMMARY

The present subject disclosure describes various embodiments for a red-light IR therapeutic element. In a first embodiment, a compact IR nipple shield device and system is provided. The compact IR nipple shield device has a cover, an arrangement of IR LED's, a flexible circuit board and a flexible overmold layer attached to the cover in between which the flexible circuit board is disposed. The flexible circuit board is electrically connected to the IR LED's and provides various routine commands thereto. The flexible overmold layer secures the flexible circuit board to the cover. The flexible circuit board and the IR LED's are secured between the cover and the flexible overmold layer. The IR nipple shield cover is adapted to be secured over a woman's nipple and the IR LED's illuminate therapeutic red light onto the woman's nipple for healing purposes.

Aspects of the disclosure are directed to a compact infrared (IR) nipple shield system. In some examples, the compact infrared (IR) nipple shield system includes a nipple shield device. In some examples the nipple shield device include a cover, an IR light-emitting diode (LED), a flexible circuit board electrically connected to the IR LED, and a flexible overmold layer connected to the cover, wherein the flexible circuit board and the IR LED are disposed between the cover and the flexible overmold layer, and wherein the cover is adapted to be secured over a woman's nipple and cause the IR LED to illuminate IR light onto the woman's nipple. In some examples, the compact IR nipple shield system includes a charger associated with the nipple shield device.

Aspects of the disclosure are directed to a compact infrared (IR) nipple shield device. In some examples, the compact IR nipple shield device includes: a cover, an IR light-emitting diode (LED), a flexible circuit board electrically connected to the IR LED, and a flexible overmold layer connected to the cover, wherein the flexible circuit board and the IR LED are disposed between the cover and the flexible overmold layer, and wherein the cover is adapted to be secured over a woman's nipple and cause the IR LED to illuminate IR light onto the woman's nipple.

Aspects of the disclosure are directed to a method of applying therapeutic infrared (IR) light to injured skin. In some examples, the method includes: securing, over a woman's nipple, a conically-shaped nipple shield device comprising: a cover adapted to be secured over the woman's nipple, an IR light-emitting diode (LED), a flexible circuit board electrically connected to the IR LED, and a flexible overmold layer connected to the cover wherein the flexible circuit board and the IR LED are disposed between the cover and the flexible overmold layer. In some examples, the method includes illuminating the woman's nipple with IR light from the IR LED. The system of the present subject disclosure further describes and includes a compact wireless charger for use with the IR nipple shield device. The compact wireless charger has a base with a cavity adapted to receive a portion of the IR nipple shield device. A power source is disposed within the compact wireless charger that is adapted to recharge a battery source disposed within the IR nipple shield device. And, a circuit board within the compact wireless charger is adapted to provide various routines to recharge the IR nipple shield device based on various parameters.

### BRIEF DESCRIPTION OF THE DRAWINGS

Various exemplary embodiments of this disclosure will be described in detail, wherein like reference numerals refer to identical or similar components or steps. The accompanying drawings, which are incorporated in and constitute a part of this specification, illustrate embodiments of the subject disclosure and technical data supporting those embodiments, and together with the written description, serve to explain certain principles of the subject disclosure. With reference to the following figures, wherein:

FIG. 1 is a front view of a compact IR nipple shield device placed over a woman's breast, according to an exemplary embodiment of the present subject disclosure.

FIG. 2 is a side view of the compact IR nipple shield device placed over the woman's nipple, according to another exemplary embodiment of the present subject disclosure.

FIG. 3 is a front view of the compact IR nipple shield device.

FIG. 4 is a rear view of the compact IR nipple shield device.

FIG. 5 is a top view of the compact IR nipple shield device illustrating the power button.

FIG. 6 is a bottom view of the compact IR nipple shield device illustrating the charging leads.

FIG. 7 is a left side view of the compact IR nipple shield device.

FIG. 8 is a right side view of the compact IR nipple shield device.

FIG. 9 is a cross-section view of the compact IR nipple shield device.

FIG. 10 is an exploded view of the compact IR nipple shield device.

FIG. 11 is a bottom view of the housing cover of the compact IR nipple shield device.

FIG. 12 is a top view of the circuit board of the compact IR nipple shield device.

FIG. 13 is a bottom view of the circuit board of the compact IR nipple shield device.

FIG. 14 is a top view of the silicon layer of the compact IR nipple shield device.

FIG. 15 illustrates an exemplary perspective view embodiment for a charging case for the IR nipple shield device, according to an exemplary embodiment of the present subject disclosure.

FIG. 16 shows a front view of the charging case with the IR nipple shield device positioned therein.

FIG. 17 shows a side view of the charging case with the IR nipple shield device positioned therein.

FIG. 18 shows a side cross section view of the charging case with the IR nipple shield device positioned therein.

FIG. 19 is a simplified electronic block diagram of an IR nipple shield system including an IR nipple shield operating in combination with a charging case, according to an embodiment of the present disclosure.

FIG. 20 depicts another exemplary embodiment integrating a portable IR nipple shield unit into a garment.

FIG. 21 shows placement of the portable IR nipple shield unit within the garment.

FIG. 22 illustrates an internal view of an LED pattern of the portable IR nipple shield unit directed to the wearer of the garment.

FIG. 23 show a perspective view of another exemplary embodiment integrating IR nipple shield unit into a portable electronic breast pump, according to an embodiment of the present disclosure.

FIG. 24 shows a rear view of the portable electronic breast pump.

FIG. 25 shows a cross section side view of the portable electronic breast pump.

FIG. 26 shows a front view of the portable electronic breast pump.

FIG. 27 show a side partial cross section view of pump insert for a breast pump attached to a breast for yet another exemplary embodiment integrating the IR nipple shield unit into a flange of the breast pump, according to an embodiment of the present disclosure.

FIG. 28 is a partial side cross section view of the pump insert.

FIG. 29 is a perspective view of the pump insert.

FIG. 30 is a side cross section view of the pump insert.

FIG. 31 is a front view of the pump insert.

FIG. 32 is a rear view of the pump insert.

FIG. 33 is a top view of the pump insert.

FIG. 34 is a side view of the pump insert.

FIG. 35 is a bottom view of the pump insert.

FIG. 36 shows a partial side cross section view of a manual breast pump attached to a breast integrating an IR nipple shield portion into a flange of the manual breast pump, according to an embodiment of the present disclosure.

FIG. 37 depicts a side cross section view of the manual breast pump integrated with the IR nipple shield portion.

FIG. 38 shows a front perspective view of the manual breast pump integrated with the IR nipple shield portion.

FIG. 39 shows an upper side perspective view of the manual breast pump integrated with the IR nipple shield portion.

FIG. 40 is an upper perspective view of the manual breast pump integrated with the IR nipple shield portion.

FIG. 41 is a side view of a closed compact charger case.

FIG. 42 is a front view of the closed compact charge case.

FIG. 43 is a perspective view of the closed compact charger case.

FIG. 44 is a side view of an opened compact charger case.

FIG. 45 is a perspective view of the opened compact charger case.

FIG. 46 is another perspective view of the opened compact charger case.

FIG. 47 is another perspective view of the opened compact charger case.

FIG. 48 is an exploded perspective view illustrating elements of the opened compact charger case.

FIG. 49 is a front view of a compact charger case.

FIG. 50 is a side cross section view of the compact charger case.

FIG. 51 is a bottom view of the compact charger case.

FIG. 52 is a top view of the compact charger case.

FIG. 53 is a perspective view of the compact charger case.

### DETAILED DESCRIPTION

The following detailed description references specific embodiments of the subject disclosure and accompanying figures, including the respective best modes for carrying out each embodiment. It shall be understood that these illustrations are by way of example and not by way of limitation.

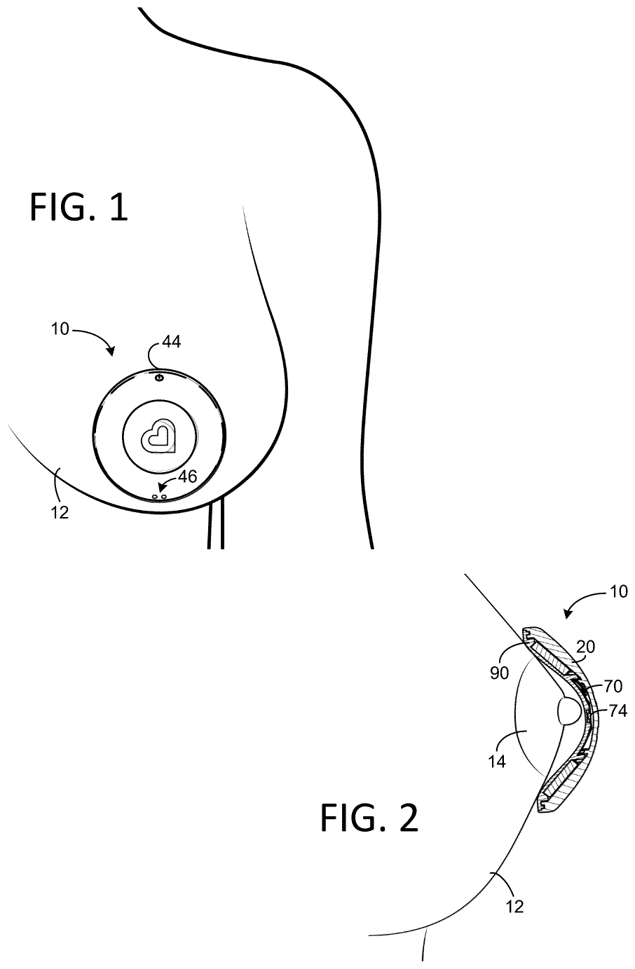

Particular embodiments of a compact red-light Infrared (IR) therapeutic element is embodied as a compact IR nipple shield 10 that will now be described in greater detail with reference to the figures. FIGS. 1 and 2 illustrate a front view, and a side cross section view of the IR nipple shield 10 placed over a woman's breast 12, and in particular to cover a nipple 14, according to an exemplary embodiment of the present subject disclosure.

FIG. 2 shows the IR nipple shield 10 having a cover housing 20, at least one infrared light emitting diode (LED) 74, a flexible circuit board 70 and a flexible overmold layer 90 attached to the cover housing 20. Although shown circular in shape, the IR nipple shield 10 may take any size or shape suitable to cover a user's breast and nipple. Likewise, although shown as a compact IR nipple shield 10, it is to be understood that the compact red light IR therapeutic element can be embodied in a variety of different configurations as will be described later.

The IR nipple shield 10 is adapted to be secured over a woman's nipple 14, and the LED 74 is adapted to illuminate a therapeutic red light onto the woman's nipple 14 for healing purposes. The red-light wavelength will be within the infrared spectrum preferably in the range between 630 and 700 nanometers (nm). At these low levels, the red light will be adapted to provide healing to the woman's nipples and breast that have become sore, cracked and/or injured during breastfeeding. The red light will promote production of mitochondria in cells to improve cellular function and repair.

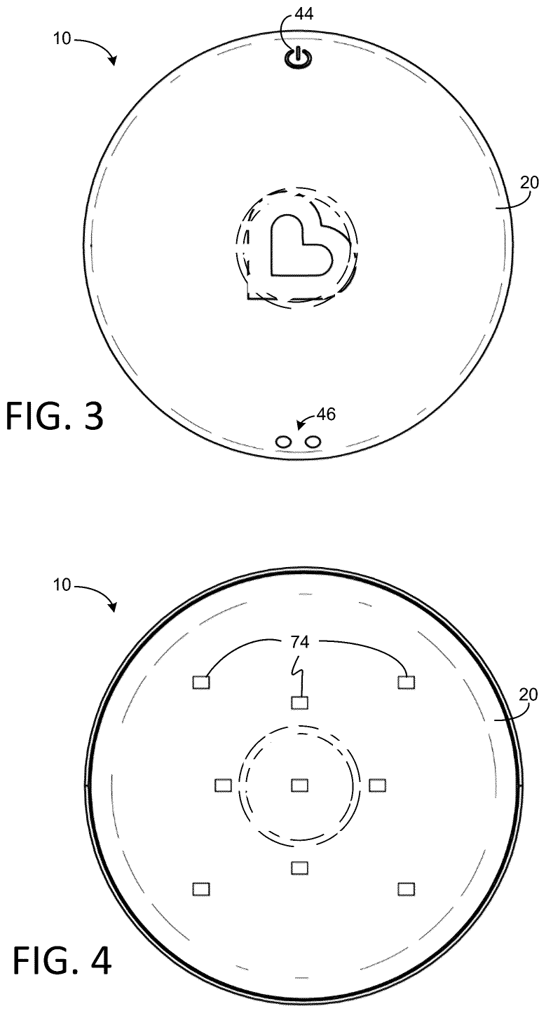

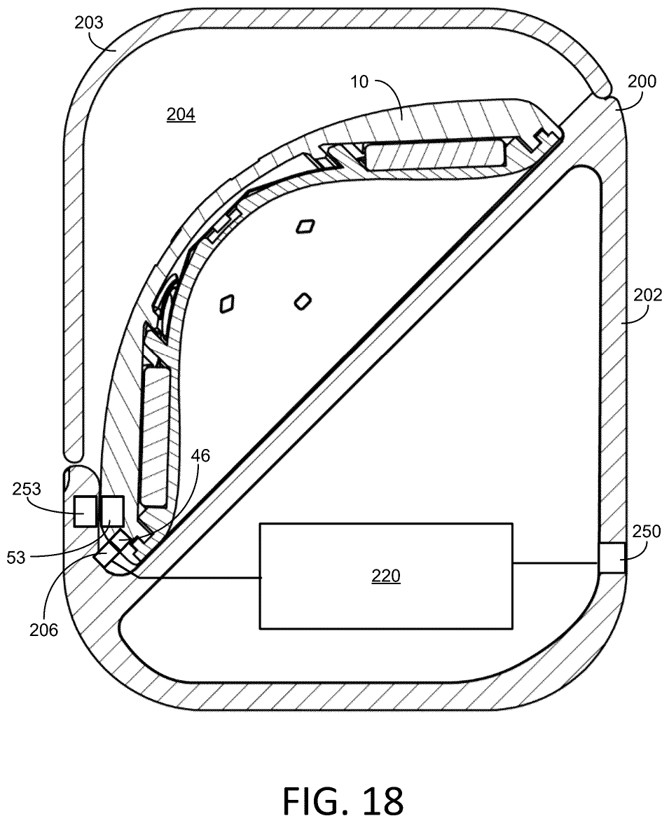

FIG. 3 shows a top view of the IR nipple shield 10. The IR nipple shield 10 includes a power button 44 to turn the IR nipple shield 10 on and off. The IR nipple shield 10 also shows a pair of battery charging electrode contacts 46 which are adapted to come into contact with a pair of mating contacts 206 (as shown in FIG. 18 and discussed in more detail later) mutually arranged to transfer a charge through the electrode contacts 46 on the IR nipple shield 10.

FIG. 4 shows a rear view of the IR nipple shield 10. As shown, various light LED's 74 are arranged to provide illumination coverage over the nipple 14 of the woman's breast 14. The arranged array of LED's 74 may be configured in any number of different ways to provide optimum coverage to a target area of the breast 12, particularly the nipple 14. Any suitable LED pattern or arrangement is possible according to this subject disclosure.

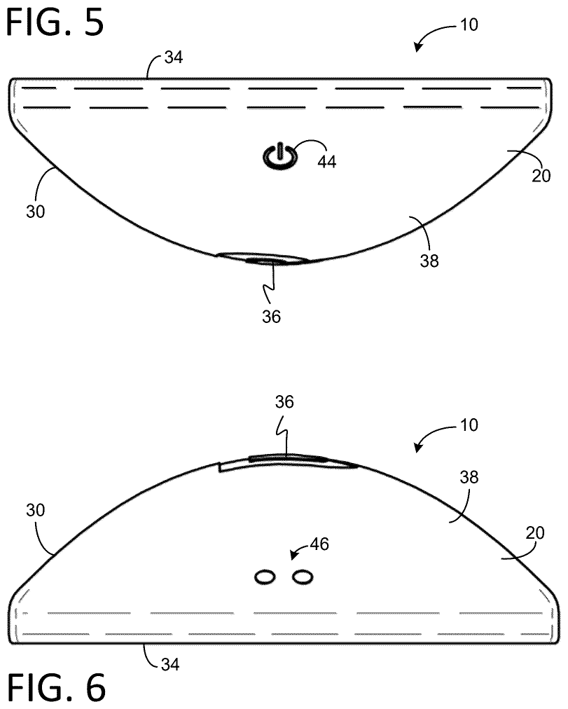

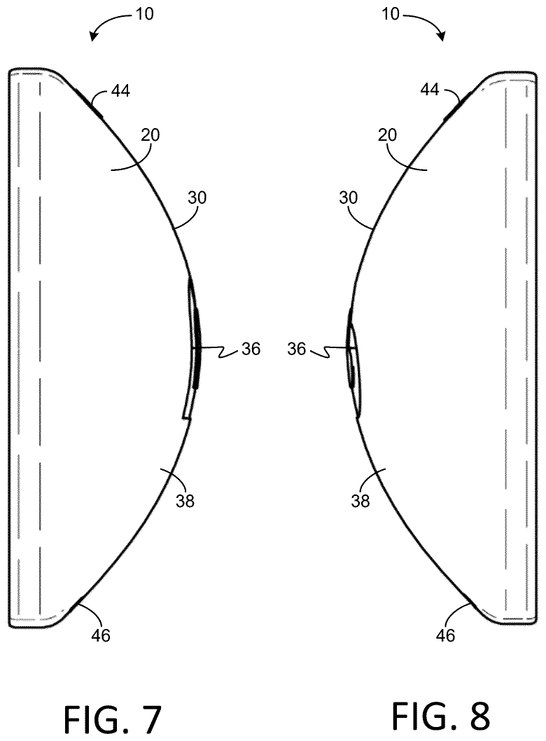

FIGS. 5 and 6 show top and bottom views respectively of the IR nipple shield device 10. FIGS. 7 and 8 depict left and right-side views of the IR nipple shield 10. As shown in FIGS. 5-8, an outer surface 38 of the outer housing 30 of the IR nipple shield 10 may take a dome shape configuration having a central apex 36 and a flat base 34 adapted to mimic the nipple end shape of a woman's breast. However, it is to be understood that various other suitable shapes and configurations are possible.

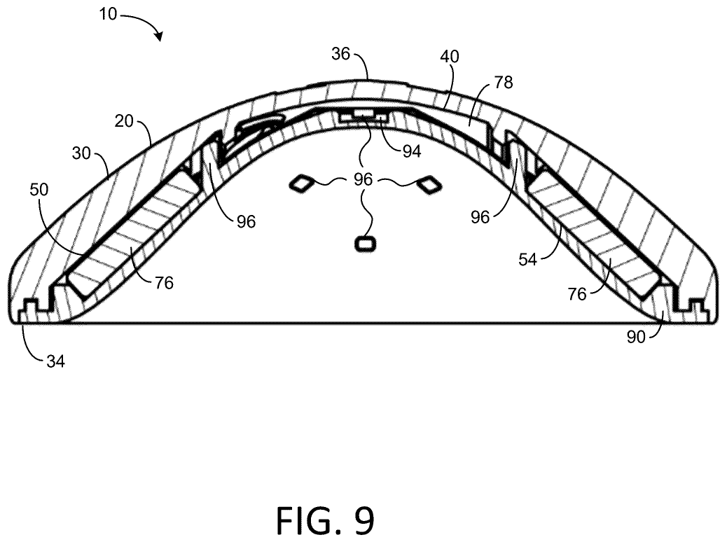

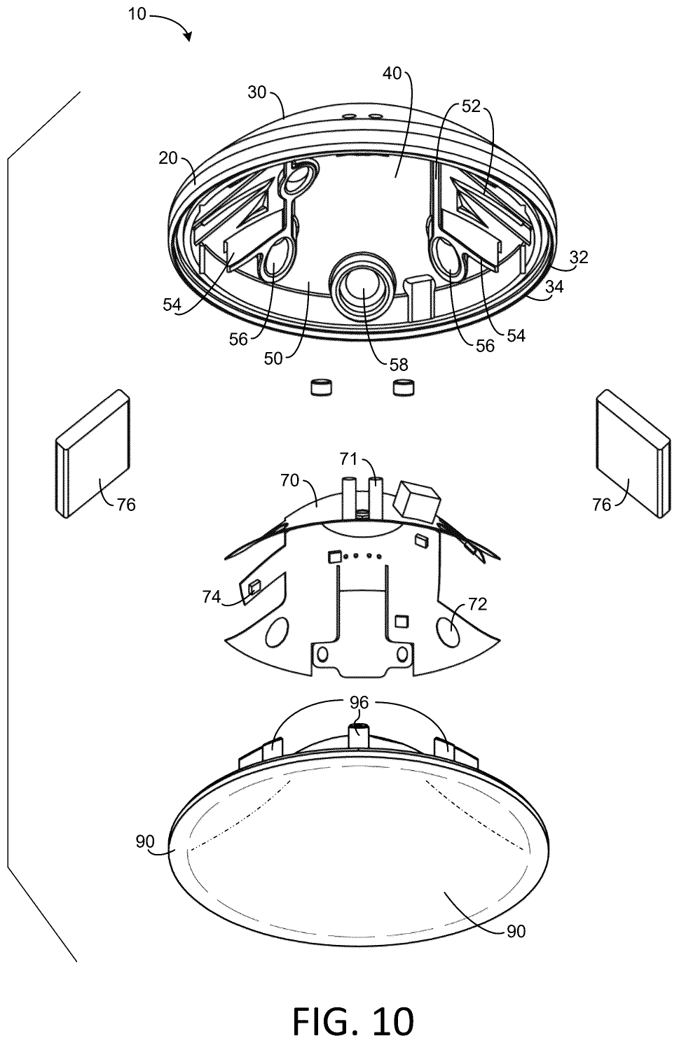

FIG. 9 shows a cross-section view of the IR nipple shield 10 in assembly construction. FIG. 10 depicts an exploded view of the IR nipple shield 10. As shown in FIG. 10, the housing cover 20 is removed from the flexible overmold layer 90. A circuit board 70 is shown disposed between the housing cover 20 and the flexible overmold layer 90. A pair of batteries 76 are shown removed away from a battery compartment 54. The battery compartment 54 is shown in assembly in FIG. 9.

As shown in FIG. 9, the batteries 76 are positioned toward an outermost portion of the housing cover 20 so that the weight of the batteries do not cause most of the gravitational force to be located toward the apex center of the IR nipple shield 10. Having a greater weight toward the apex of the IR nipple shield 10 may create a larger cantilevered downward force encouraging the IR nipple shield 10 to fall from the mothers breast when it is position for use.

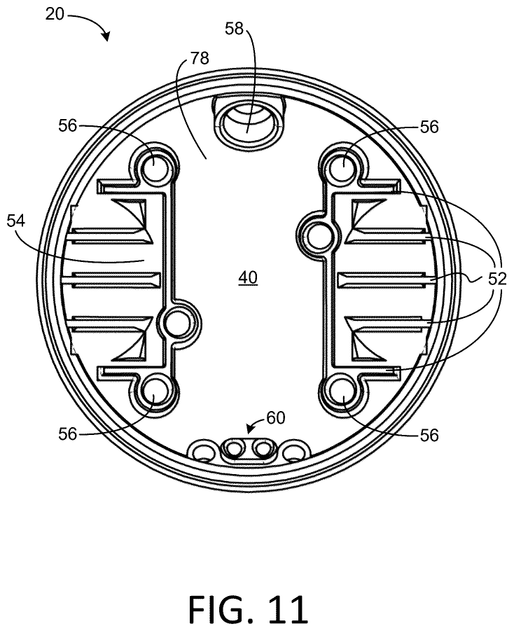

FIG. 11 illustrates a bottom view of the housing cover 20. The housing cover 20 shows various sockets 56 adapted to receive and secure posts 96 from the flexible overmold layer 90 during assembly. The housing cover 20 includes various ribs 52 provided to reinforce the thin construction of the housing cover 20 and to provide structure and open spaces for various components and the circuitry connected within and disposed on the circuit board 70. The ribs 52 can be used as walls to secure the batteries 76 in position within the housing cover 20. Likewise, the ribs 52 can be provided to elevate the circuit board 70 away from the inner surface 40 a predetermined distance from the housing cover 20 to define an electronic storage compartment 78 that allows for room for the various components on the flexible circuit board 70.

The housing cover 20 may include a power switch cavity 58 into which an on/off switch 44 and components thereof may be located. Likewise, the housing cover 20 may also include an electrode contact cavity 60 into which the battery charging electrode contacts 62 components may be located.

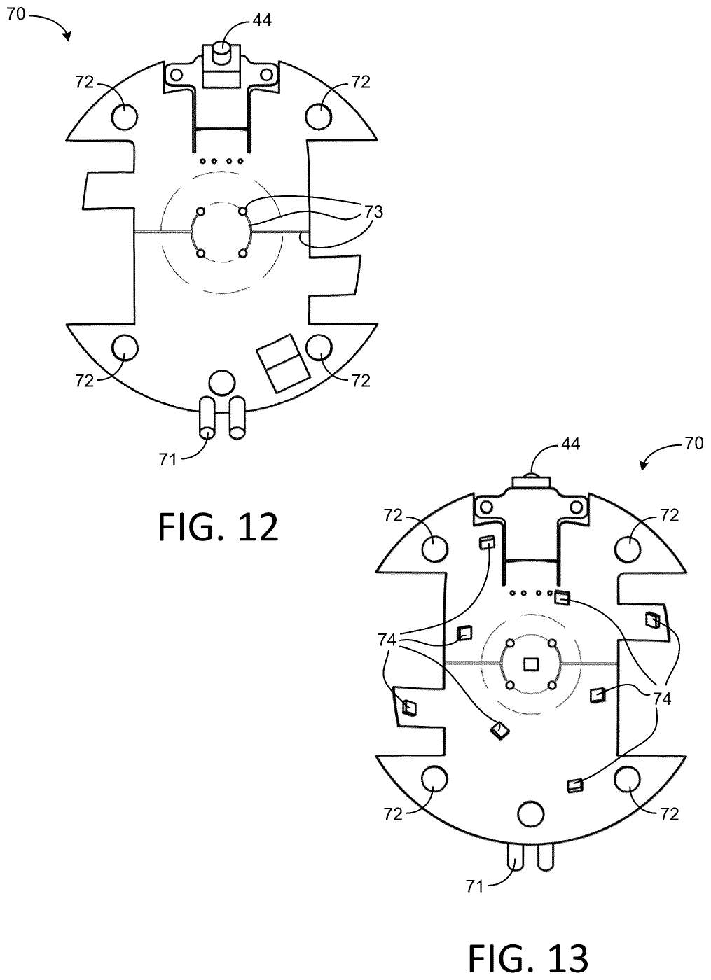

FIGS. 12-13 show a top and a bottom view of the flexible circuit board 70. The top view of the flexible circuit board 70 would contain most of the various components of the circuit adapted to electrically connect the LED's 74 to a power source. The various circuit components disposed on the top end of the flexible circuit board 70 would be conveniently housed within the electronic compartment 78 defined between the inner surface 40 of the housing cover 20 and the upper side of the circuit board 70.

The circuit board 70 is a thin flexible wafer upon which the various electronic circuitry is connected. To allow for the flexibility, the circuit board 70 may have slits or perforations 73 to allow for the circuit board 70 to bend in different positions within the housing cover 20.

An on/off switch 44 is provided to initiate power to the IR nipple shield 10. The on/off switch 44 is accessible from outside of the housing cover 20 as shown in FIGS. 1 and 3. Various different types of on/off switches may be employed according to this subject disclosure.

A pair of electrode posts 71 are provided to electrically connect the circuit board 70 to the electrodes 62 positioned within the housing cover 20. The electrode posts 71 can also be provided to align the electrodes 62 within the electrode cavity 60 in a secure position between the housing cover 20 and the flexible overmold layer 90.

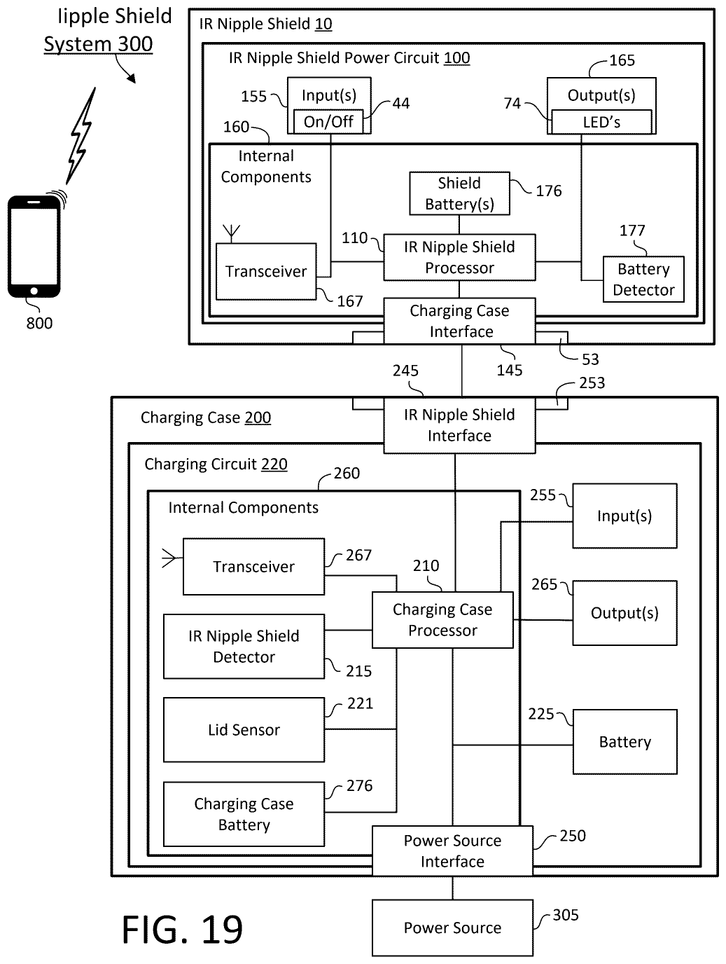

It is to be understood that various elements of the circuit board 70 are electrically connected as an IR nipple shield power circuit 100 (shown in FIG. 19) operable to illuminate the LED's 74 within the IR nipple shield 10. Likewise, as will be described later, the IR nipple shield power circuit 100 is adapted to recharge the batteries 76 in the IR nipple charger 10.

In FIG. 13, the bottom view of the flexible circuit board 70 illustrates various LED's 74 disposed throughout the surface of the circuit board 74. The LED's are angled and positioned in an array to optimize red light illumination onto the users skin. On the circuit board 70, various apertures 72 are provided to be aligned with and receive the overmold posts 96 extending from the flexible overmold layer 90. Although the alignment posts are shown extending from the overmold layer, it is to be understood that various different types of alignment mechanisms may be employed in order to assembler the IR nipple shield.

The circuit board 70 is obtusely shaped to optimally fit between the housing cover 20 and the overmold layer 90. As shown in FIG. 13, the LEDs 74 are arranged substantially symmetrical around the IR nipple shield 10 to shine an even pattern of red light over the skin of the user during use.

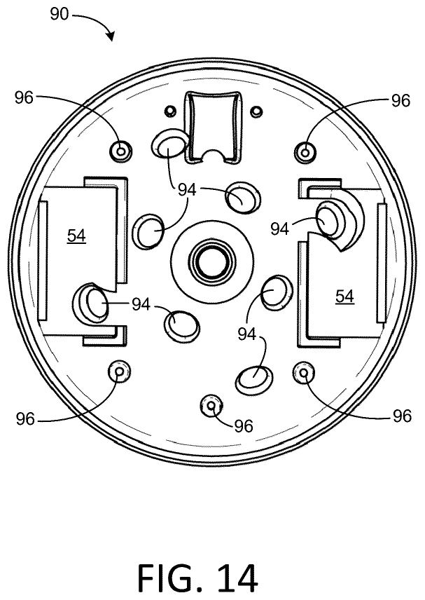

FIG. 14 is a bottom view of the flexible overmold layer 90. Various blind holes 94 are disposed in the flexible overmold layer 90. The blind holes 94 are aligned and positioned to receive the various LED's 74. As shown in FIG. 9, a predetermined depth has been provided in the blind holes 94 to allow the LED's to be placed closer to an outer surface of the flexible overmold layer 90 allowing for the greatest amount of red-light emission from the LEDs 74.

The flexible overmold layer 90 also includes ribs 98 to position and secure the batteries 76 and other components within the IR nipple shield 10. Various posts 96 are shown extending from the overmold layer 90 that are adapted to align and secure the flexible circuit board 70 between the housing cover 20 and the overmold layer 90.

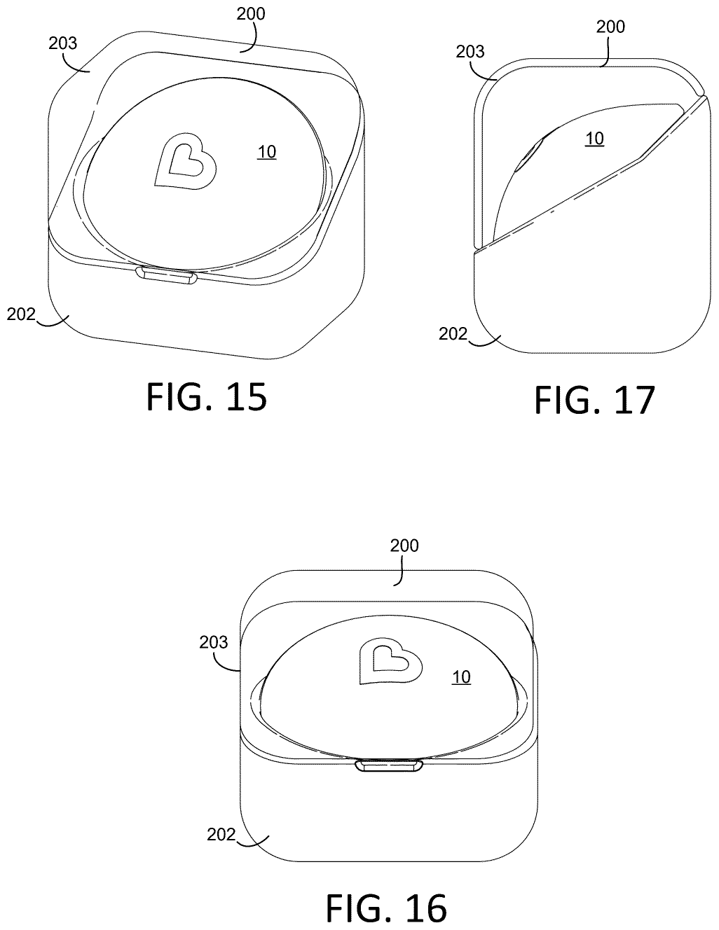

FIGS. 15-17 illustrate an exemplary embodiment for a charging case 200 adapted to receive and recharge the batteries 76 for a compact red light IR therapeutic element such as the IR nipple shield 10. As shown in FIGS. 15-18, the charging case 200 is adapted to secure the IR nipple shield 10 within the housing 202 of the charging case 200.

In FIG. 18, an internal cavity 204 is shown constructed within the housing 202 that is adapted to receive at least a portion of the IR nipple shield 10 to be conveniently aligned, stored and charged simultaneously. The lower end of the internal cavity 204 depicts a mating pair of electrode contacts 206 adapted to mate with the electrode contacts 46 on the IR nipple shield 10. The charging case 200 includes a charging circuit 220 adapted to perform various functions according to the subject disclosure as will be described later. In use, at least one function is that the charging case 200 will charge the batteries 76 provided in the IR nipple shield 10 while the IR nipple shield is docked within the charging case 200.

As shown in FIG. 18, during placement and alignment of the IR nipple shield 10 within the charging case 200, one or more retention magnets and/or magnetic material 53 may be placed within the IR nipple shield 10. Likewise, one or more mating retention magnets and/or magnetic material 253 may be placed within the charging case 200 to allow the magnets 53 of the IR nipple shield 10 to be easily oriented and properly aligned with the magnets 253 within the charging case 200 so that the electrode contacts 46 on the IR nipple shield 10 are properly aligned with the electrode contacts 206 on the charging case 200.

It is to be understood that the charging case 200 and/or the cavity 204 may take any number of configurations according to this subject disclosure. Embodiments of the disclosure are not limited to any particular shape and can have different shapes to accommodate different configurations.

The charging case 200 includes a lid 203 attached to the housing 202 of the charging case 200. The lid 203 may be operable between a closed position where the lid 203 is aligned over the cavity 204 fully enclosing the IR nipple shield 10 within the housing 202. The lid 203 may be positioned in an open position where the lid 203 is displaced and/or pivoted away from the housing 202 and the cavity 204 such that a user can remove the IR nipple shield 10 from the cavity 204 or replace the IR nipple shield 10 within the cavity 204. The lid 203 can be pivotably attached to the housing 202 and can include a magnetic or mechanical closing system.

FIG. 19 is a simplified electronic block diagram of an IR nipple shield system 300. The IR nipple shield system 300 includes a combination of an IR nipple shield 10 operating in combination with a charging case 200. The IR nipple shield 10 includes an IR nipple power circuit 100 including circuitry adapted to power LED's to provide healing therapy to an affected skin area. The charging case 200 includes a charging circuit 220 including circuitry adapted to recharge the IR nipple shield 10. The IR nipple power circuit 100 is electronically connected to the charging circuit 220 according to an embodiment of the present disclosure.

The IR nipple shield 10 includes at least one shield battery 76 configured to power the IR nipple shield 10, and the charging case 200 includes a case battery 276 configured to power the charging case 200. The case battery 276 is also adapted to recharge the shield battery 76 when the shield battery 76 is low. Recharging of the shield battery 76 can take place when the IR nipple shield 10 is docked within the charging case 200 or within a predetermined proximity to receive the transfer of a charge from the charging case 200.

The IR nipple shield power circuit 100 includes a circuit board 70 having electronic connectivity for various components, including but not limited to, various inputs, outputs, internal components and a processor having storage, routines, memory, detectors, a transceiver, interfaces and/or any other suitable component capable of operating the IR nipple shield 10 according to this subject disclosure.

The IR nipple shield power circuit 100 in the IR nipple shield 10 can each have one or more inputs 155. Various inputs 155 may include, for example, an on/off switch, audible or tactile switches that detect a user's touch or verbal command. Other inputs may include an accelerometer or a capacitive sensor for activation, for example, by a user to activate the LEDs or command the IR nipple shield 10 to enter a pairing mode that can be indicated by a light on the IR nipple shield 10.

The IR nipple shield power circuit 100 may embody one or more internal components 160. The various internal components 160 may include, for example, a battery detector 177, a speaker, a microphone, indicator lights, a rechargeable battery, other detectors, a processor, and/or other circuitry and internal components 160. The battery detector 177 can monitor the battery level in the shield battery 176.

The IR nipple shield power circuit 100 may embody one or more outputs 165. The various outputs may include, for example, an IR light from the LEDs, a speaker, indicator lights, and/or other indicators. The indicator light can indicate a battery charge level or state, a pairing mode and/or other function suitable to operation of the IR nipple shield 10.

The IR nipple shield 10 can include a wireless transceiver 167 that can operate both as an input 155 or an output 165 device. That is, the wireless transceiver 167 can enable the IR nipple shield 10 to send or receive input communication signals from a device (such as the charging case 200, a smart device 800 through the IR nipple shield system 300 to a mobile phone, tablet, computer, etc.) that can send an instruction command to the IR nipple shield 10 or charging case 200 to perform a predetermined action.

The wireless transceiver 167 of the IR nipple shield 10 may include a communication array that may include a transmitter, transducer, an antenna, and may be adapted for communication using any of a variety of custom or standard wired (Ethernet, LAN, HomePlug, etc.) or wireless protocols, such as: IEEE 802.15.4, Wi-Fi, ZigBee, infrared, LTE, ultraband, 6LoWPAN, Thread, Z-Wave, radio, Bluetooth Smart, WirelessHART, MiWi and/or including any other suitable communication protocols.

IR nipple processor 110 can be configured to control various functions of the IR nipple shield 10 as described in more detail below. In some embodiments, a shield detector 215 can include one or more sensors that detect when the IR nipple shield 10 is placed within charging case 200. The shield detector 215 can be any type of mechanical or electrical sensor, such as, but not limited to, a magnetic sensor, an optical sensor, a switch, a hall effect sensor, a flux sensor, a capacitive sensor, a photodetector, a proximity detector, a momentary switch or any other type of sensor or combination of the above listed sensors

The IR nipple shield 10 may also include a controller, an integrated CPU, printed circuit board, at least one circuit, a power supply, at least one USB port, a timer and memory for storing programs, routines and data. The processor 110 may be implemented with a general-purpose computer having a printed circuit board, at least one circuit, central processing unit, a power supply, at least one USB port, a timer and memory for storing programs, routines and data and a bus connecting the various components (input devices, output devices, internal components, interfaces, and the like).

The IR nipple shield 10 includes a charging case interface 145. The charging case interface 145 electrically connects with an IR nipple shield interface 245 disposed in the charging case circuit 220 within the charging case 200. The connection between the charging case interface 145 and the IR nipple shield interface 245 enables the charging case circuit 220 within charging case 200 to communicate with and/or charge the IR nipple shield 10 based on a battery charge level detection from the battery detector 177 in the IR nipple shield Power circuit 100.

Although shown as electrode contacts 46, 206 (FIGS. 3 and 18) provided on the charging case interface 145 and the IR nipple shield interface 245 respectively, it is to be understood that various electrical connections are possible. That is, the communication between the interfaces 145, 245 can be a physical connection, such as pairing electrical connectors (e.g., electrode pairs 46 and 206 in FIG. 18), or a wireless power transmitter, with wireless power receiving coils, that can receive inductive power from a battery 225 in the charging case 200 or some other power source 305. The interfaces 145, 245 can transfer power and/or data between the charging case 200 and the IR nipple shield 10.

The charging case circuit 220 includes a case processor 210 having electronic connectivity for various components, including but not limited to, various inputs, outputs, and internal components, a processor having storage, routines, memory, detectors, a transceiver, interfaces and/or any other suitable component capable of operating the charging case 200 and the IR nipple shield 10 according to this subject disclosure.

As to the charging case 200, the charging case circuit 220 in the charging case 200 can have one or more inputs 255. The various inputs 255 may include, for example, an on/off switch, tactile switches that detect a user's touch or an opening of the lid 203. Other inputs may include an accelerometer or a capacitive sensor for activation, for example, by a user to activate the LEDs when the IR nipple shield 10 is removed from the charging case 200. Removal of the IR nipple shield 10 from the charging case 200 may trigger the IR nipple shield 10 and the charging case 200 to enter into a pairing mode so that the IR nipple shield 10 can communicate with the charging case 200 during use when it is away from the charging case 200.

The charging case circuit 220 may embody one or more internal components 260. As described in more detail below, the various internal components may include, for example, a speaker, a microphone, indicator lights, a rechargeable battery, a processor, sensors, detectors, transceiver and/or other circuitry and internal components within the charging case circuit 220.

The charging case circuit 220 within the charging case 200 may embody one or more outputs 265. The various outputs may include, for example, LEDs, a speaker, indicator lights, and/or other indicators. The indicator light can indicate a battery charge level, a pairing mode and/or other function suitable to operation of the IR nipple shield 10.

The charging case circuit 220 can also include a wireless transceiver 267 that can operate both as an input 255 and an output 265 device. The wireless transceiver 267 can communicate with the IR nipple shield 10 and/or a remote smart device 800 through the IR nipple shield system 300, such as to a mobile device, a tablet, a computer or the like. The wireless transceiver 67 can communicate data and/or other signals, or commands to and from the IR nipple shield 10 and/or a remote smart device 800. Signal commands and/or other data communication about the IR nipple shield 10 can come to and/or from the smart device 800.

The wireless transceiver 267 of the charging case 200 may include a communication array that may include a transmitter, transducer, an antenna, and may be adapted for communication using any of a variety of custom or standard wired (Ethernet, LAN, HomePlug, etc.) or wireless protocols, such as: IEEE 802.15.4, Wi-Fi, ZigBee, infrared, LTE, ultraband, 6LoWPAN, Thread, Z-Wave, radio, Bluetooth Smart, WirelessHART, MiWi and/or including any other suitable communication protocols.

The charging case circuit 220 of the charging case 200 can include a charging case processor 210. The case processor 210 can be configured to control various functions of the charging case 200 and/or the IR nipple shield 10.

The case processor 210 may be implemented with a general-purpose computer having a printed circuit board, at least one circuit, central processing unit, a power supply, at least one USB port, a timer and memory for storing programs, routines and data and a bus connecting the various components (input devices, output devices, internal components, interfaces, and the like).

The charging case 200 may include a power source interface 250 that enables the charging case 200 to be electrically connected to a power source 305. The power source interface 250 may be electrically coupled to the power source 305 in a variety of different ways. The power source interface 250 may be coupled to the charging case 200 via a wired or wireless power source connection. The power connection may be an AC or DC power source, an inductive charging pad and/or any other power transmission method according to this subject disclosure.

The charging case battery 276 in the charging case 200 may be any type of suitable battery such as a rechargeable battery. In operation, the case battery 276 can be recharged by the power source 305. Likewise, the power source 305 can be electrically connected through the charging case circuit 220 and the IR nipple shield power circuit 100 to recharge the shield battery 176. In use, one or both of the power source 305 and the charging case battery 276 may be used to recharge the shield battery 176.

The power source interface 250 can be part of a receptacle connector for a micro-USB connector, a Lightening connector or other connector that can provide power to the IR nipple shield 10. Alternatively, or in addition to a receptacle connector, the power interface 250 can include a wireless power receiver, such as one or more wireless power receiving coils, that can receive inductive power from the power source 305.

The IR nipple shield interface 245 and the charging case interface 145 can transfer power and/or data bidirectionally between each other. The power source interface 250 can include various types of electrical connectors, or a wireless power transmitter, such as a wireless power transmitting coil that can transmit inductive power to an inductive power receiver within the charging case 200 from the power source 305.

An IR nipple shield detector 215 may be included in the charging case circuit 220 to detect the presence or absence of the IR nipple shield 10. When the IR nipple shield 10 is positioned within the charging case 200 and detected by the IR nipple shield detector 215, the charging case circuit 220 can detect whether the IR nipple shield 10 has a low battery level through the battery detector 177 in the IR nipple shield 10 and send command signals to automatically recharge the shield battery 176 for its next use.

A lid sensor 221 may be included as part of the charging circuit 220. If the charging circuit 220 detects via the lid sensor 221 that the lid 203 is open, the charging circuit 220 may detect whether the IR nipple shield 10 is present and is about to be removed from the charging case 200, or whether the is absent and the IR nipple shield 10 is about to be inserted into the charging case 200. The lid sensor 221 is adapted to work in coordination with the IR nipple shield detector 215 to detect if the IR nipple shield 10 is present or not within, or in an area proximally close to the charging case 200.

The case detector 215 may include one or more sensors. The various sensors may be used to, amongst various tasks, detect when the IR nipple shield 10 is placed within the charging case 200. The shield detector 215 can be a circuit that periodically “pings” the IR nipple shield contacts to determine if the IR nipple shield 10 is present. The case detector 215 can be any type of mechanical or electrical sensor, such as, but not limited to, a magnetic sensor, an optical sensor, a switch, a hall effect sensor, a flux sensor, a capacitive sensor, a photodetector, a proximity detector, a momentary switch or any other type of sensor.

In response to detecting the insertion of the IR nipple shield 10 within the charging case 200, the case detector 215 can generate a detect signal that can be sent to the battery detector 177 to detect a battery level in the IR nipple shield 10. The detection signals can be processed by other circuitry within the charging case 200 to initiate charging of the IR nipple shield 10. The case detector 215 can also be used to stop charging of the IR nipple shield 10. For example, the case detector 215 can detect when the IR nipple shield 10 is removed from the charging case 200 and generate a removal signal that stops the charging.

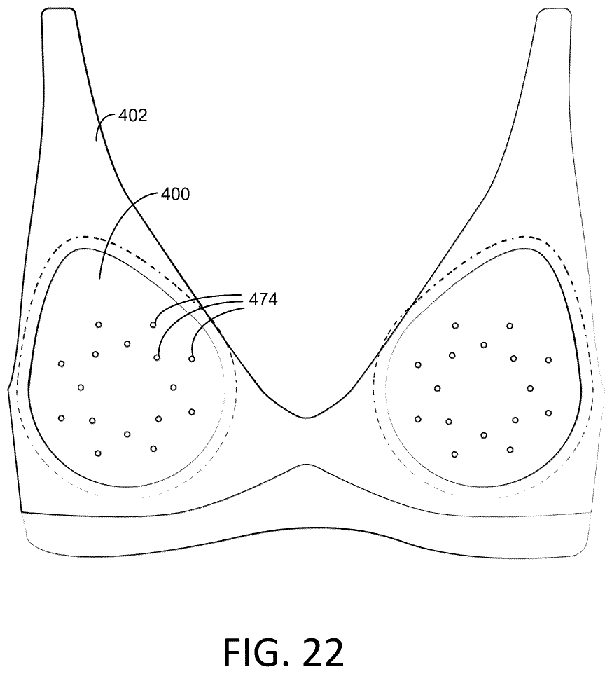

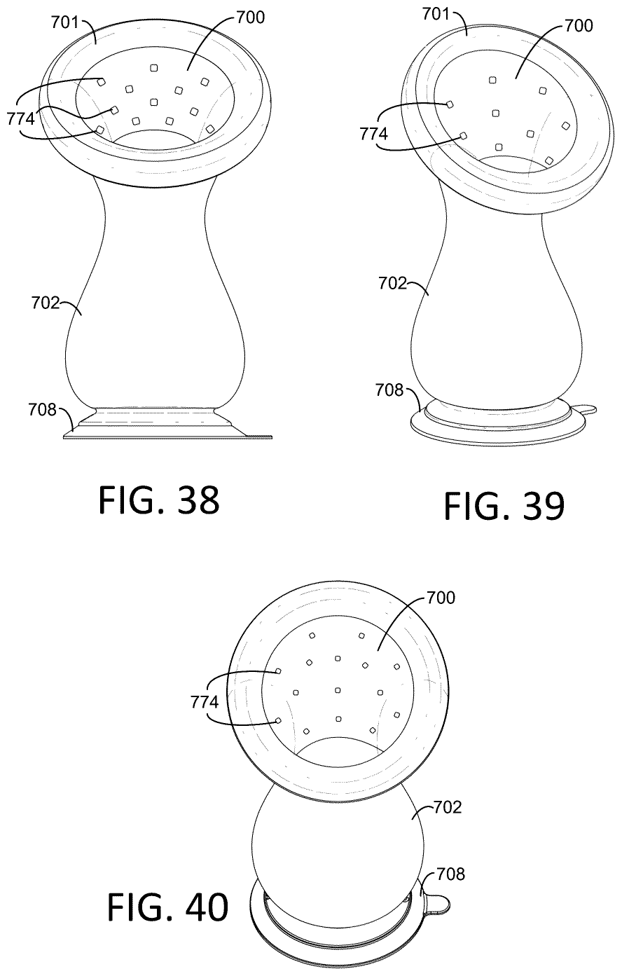

Although the compact red light IR therapeutic element is embodied as a compact IR nipple shield 10, it is to be understood that the red-light IR therapeutic element can be configured in a variety of different constructions as will be described below in FIGS. 22-40.

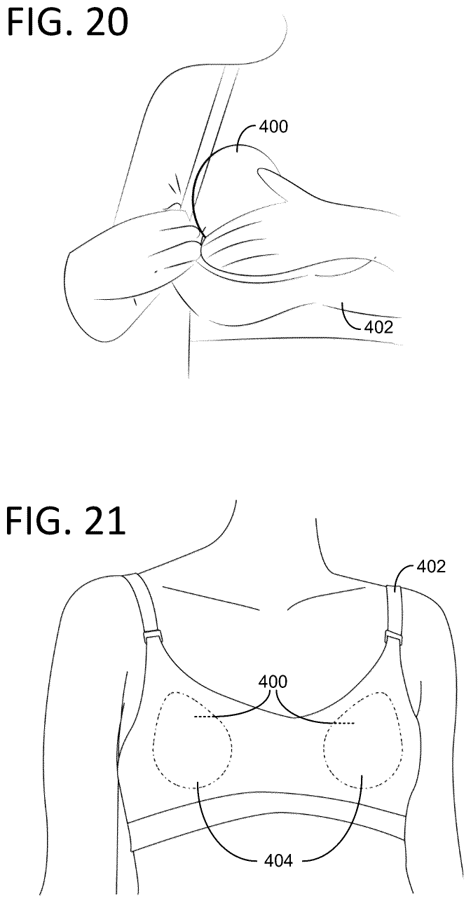

FIGS. 20-22 depict another exemplary embodiment for a red-light IR therapeutic element that integrates a compact red light IR therapeutic insert 400 into a garment 402. The compact red light IR therapeutic insert 400 is portable and can be removably located in a variety of different garments or articles of clothing. As shown, the garment 402 is a bra into which pockets 404 can be configured to receive the compact red light IR therapeutic insert 400. As shown in FIG. 22, various LEDs 474 are arranged and directed toward a woman's nipple 14 to provide optimum coverage and illumination to provide healing therapy to the injured skin areas. Although shown as a bra, it is to be understood that the garment may be any type of garment capable of securing the compact red light IR therapeutic insert 400 at a preferred location to illuminate the LED's at a desired area. The compact red light IR therapeutic insert 400 may be may of a soft flexible material such as silicone to provide comfort to the nursing mother while in use.

The compact red light IR therapeutic device includes a cover, IR LEDs 474 arranged in a pattern. A flexible circuit board is electrically connected to the IR LEDs and a flexible overmold layer is connected to the cover. The flexible circuit board and the IR LED's are disposed between the cover and the flexible overmold layer. The compact red light IR therapeutic insert 400 is adapted to be positioned within the garment 402 over, and laying against, a portion of skin and applying an illuminated red light onto the portion of skin needing therapy.

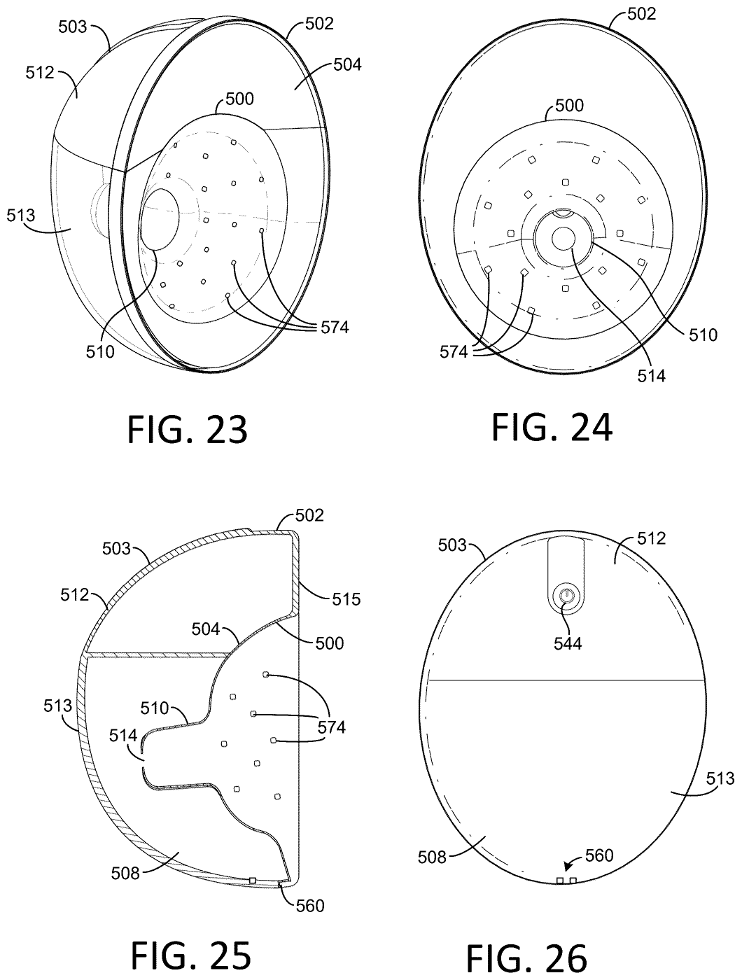

FIG. 23 shows a perspective view of another exemplary embodiment for the red-light IR therapeutic element by integrating a compact red light IR therapeutic element 500 into a portable electronic breast pump 502, according to an embodiment of the present disclosure. The portable electronic breast pump 502 has an outer shell 503. The outer shell 503 has an upper portion 512 and a lower portion 513.

The compact red light IR therapeutic element 500 is integrated into a breast cavity 504 portion within the electronic breast pump 502. The breast cavity 504 and the nipple cavity 510 of the compact red light IR therapeutic element 500 are best shown viewed from the side view in FIG. 25 and in the rear view depicted in FIG. 24. The end of the nipple cavity 510 has an opening 514 through which a mothers milk may enter and collect in the reservoir 508.

The compact red light IR therapeutic element 500 may be may of a soft flexible material such as silicone to provide comfort to the nursing mother while in use.

As shown, the outer shell 503 of the electronic breast pump 502 has a rounded shape, and a flat base 515 on a rear end to lie comfortably against a woman's breast 12. The nipple cavity 510 and the opening 514 are provided in a center of the compact red light IR therapeutic element 500 through which a nipple 14 of the breast can be placed within from which milk is expressed and flows through the opening 514 and into the reservoir 508.

The lower portion 513 is a collection reservoir 508 into which the expressed milk is collected. The reservoir 508 in the lower portion 513 may be viewed through the lower portion 513 which may be made of a translucent material.

The upper portion 512 of the electronic breast pump 502 may house the pump electronics. As before, the pump electronics are adapted to operate various LEDs 574 around a portion of the breast to be provided with therapeutic healing red light IR.

Various LEDs 574 are provided in the compact red light IR therapeutic element 500 and arranged and directed toward a woman's nipple 14 to provide optimum coverage and illumination to provide healing therapy to injured skin areas before, during or after use.

The electronic breast pump 502 may contain rechargeable batteries. The pump electronics can control recharging a rechargeable battery disposed within the compact red light IR therapeutic element 500 when electrode contacts 560 thereon are connected to a power source. An on/off switch 544 may be provided on the upper portion 512 of the outer shell of the electronic breast pump 502.

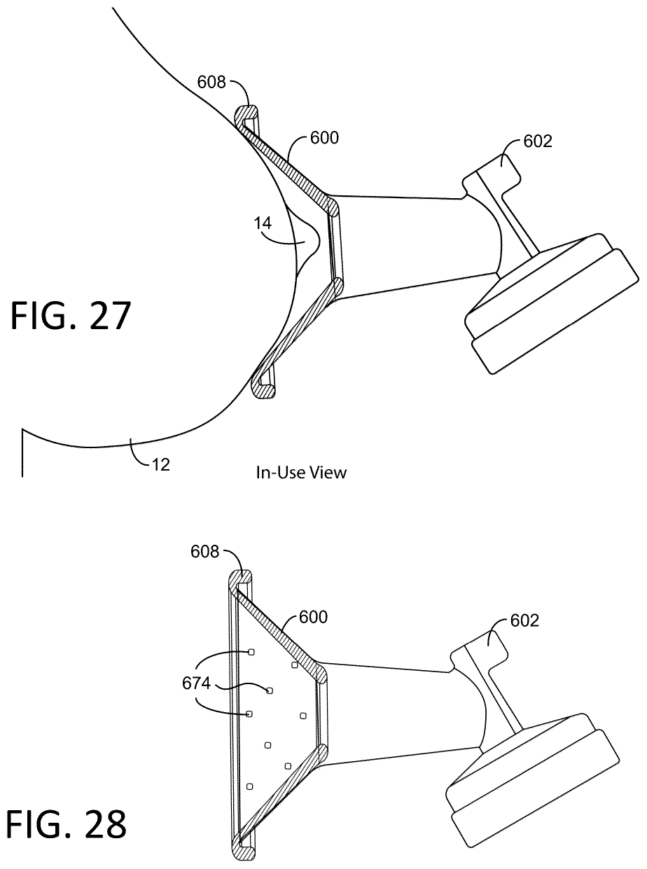

FIG. 27 shows a side partial cross section view yet another exemplary embodiment for the red-light IR therapeutic element as a breast pump flange 600 for a breast pump connector 602 attached to a woman's breast 12. FIG. 28 shows the breast pump flange 600 assembled with the breast pump connector 602. It may be removably connected, or more permanently connected to the breast pump connector 602. The breast pump flange 600 may be may of a soft flexible material such as silicone to provide comfort to the nursing mother while in use.

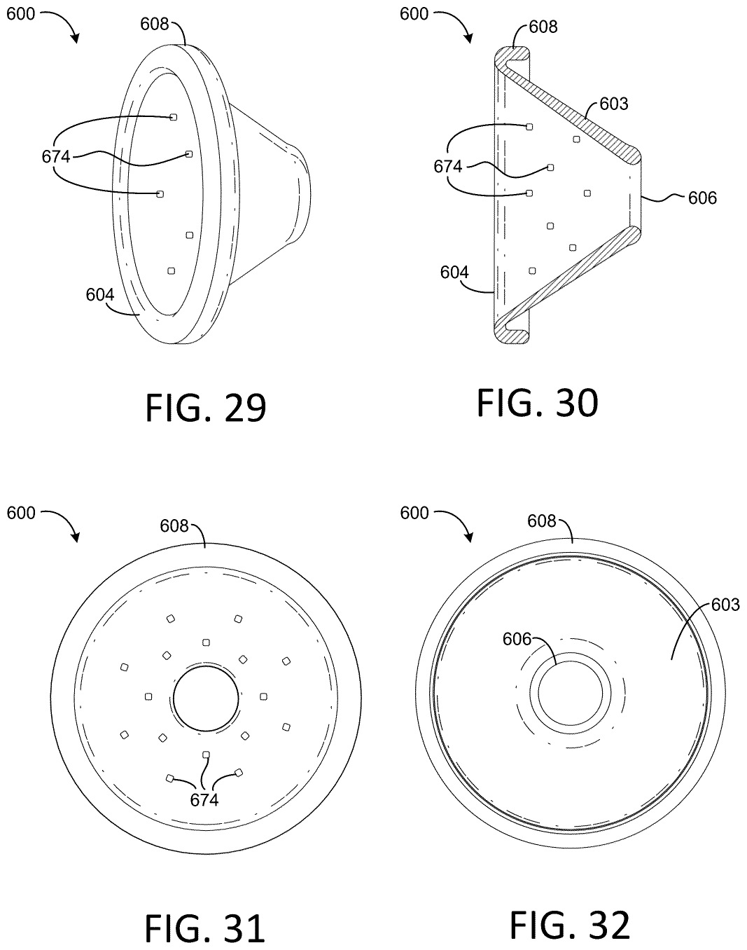

FIGS. 29-35 shows various views of the breast pump flange 600. The breast pump flange 600 has a frustoconical sidewall 603 with a first end 604 wider than a second end 606 which is narrower in shape. The first end 604 has a return lip 608 that returns backward. The second end 606 is an adapter that is connected to the breast pump connector 602.

Various LEDs 674 are provided in the breast pump flange 600 and arranged and directed toward a woman's breast 12 and nipple 14 to provide optimum coverage and illumination to provide healing therapy to injured skin areas before, during or after use.

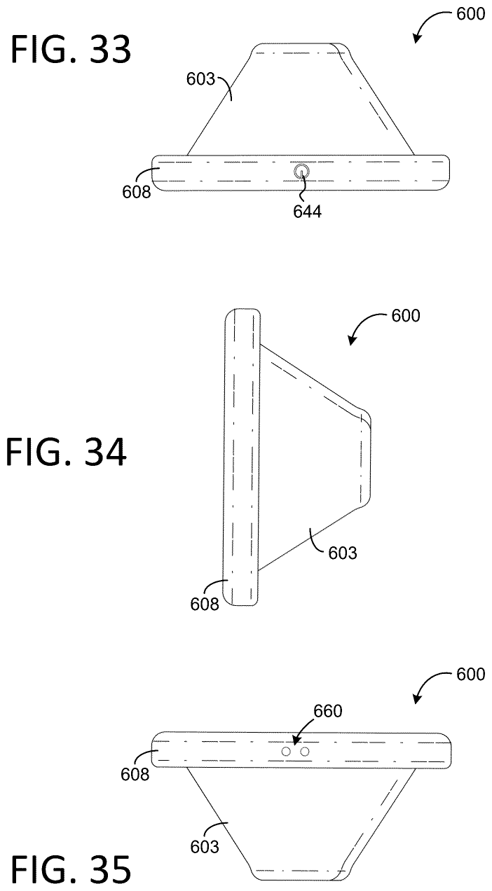

FIG. 33 shows an on/off switch 644 adapted to power on the breast pump flange 600 for use. FIG. 34 shows that the breast pump flange 600 may contain internal rechargeable batteries that can be recharged when electrode contacts 660 are connected to a power source.

As before, the breast pump flange 600 may be frustoconical shaped and attached to connector 602. The breast pump flange 600 includes a cover, a flexible overmold layer, and IR LEDs arranged in a pattern that are electronically connected to a flexible circuit. The IR LEDs are encased within the cover attached to the flexible overmold layer, and the compact red light IR therapeutic element within the frustoconical shaped flange is adapted to be positioned over a portion of the breast and to apply an illuminated red light onto the portion of breast needing therapy.

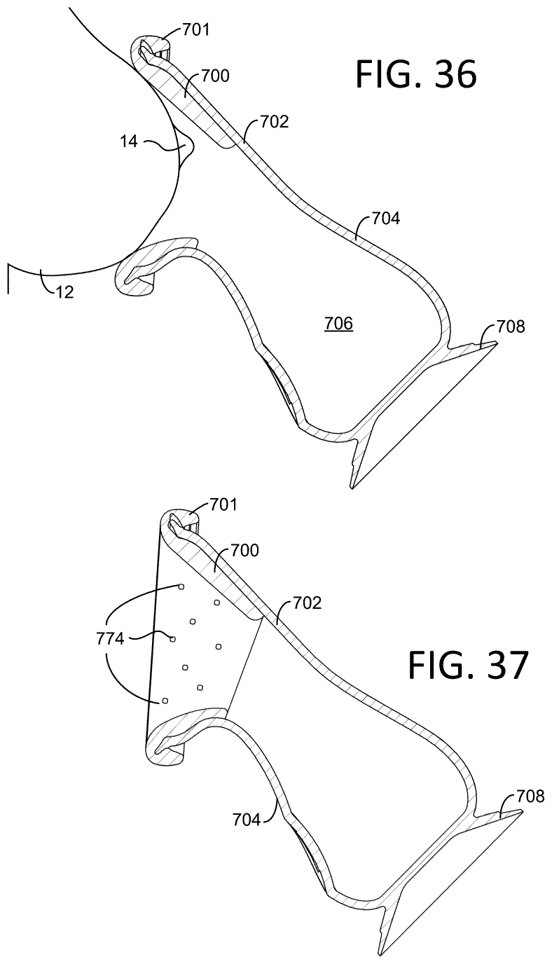

FIGS. 36-40 show various views of another embodiment for the red-light IR therapeutic element configured as an IR nipple shield flange 700 integrated into a manual breast pump 702, according to an embodiment of the present disclosure. FIG. 36 is a partial side cross section view of a manual breast pump 702 attached to a breast 14 with the IR nipple shield flange 700. In FIG. 37, the IR nipple shield flange 700 is shown integrated into an open end of the manual breast pump 702. The IR nipple shield flange 700 may be made of a soft flexible material such as silicone to provide comfort to the nursing mother while in use.

The manual breast pump 702 may be embodied as a flexible container 704 that can be compressed and expanded to provide manual pumping in order to express milk from the mother's breast 12. The manual breast pump 702 contains a collection reservoir 706 to hold the milk. A base of the manual breast pump 702 may contain a suction cup 708 in order to place the manual breast pump 702 on a flat surface and to secure it to the surface so that it does not tip over easily. The IR nipple shield flange 700 includes a return lip 701.

As shown, various LEDs 774 are provided in the IR nipple shield flange 700 and arranged and directed toward a woman's breast 12 and nipple 14 to provide optimum coverage and illumination to provide healing therapy to injured skin areas before, during or after use.

Although not shown, the IR nipple shield flange 700 may also be provided with an on/off switch adapted to power on the IR nipple shield flange 700 for use. The IR nipple shield flange 700 may also contain internal rechargeable batteries that can be recharged when electrode contacts are connected to a power source.

As before, the compact red light IR therapeutic element or IR nipple shield flange 700 has a cover, a flexible overmold layer, and IR LEDs arranged in a pattern that are electronically connected to a flexible circuit. The IR LEDs are encased within the cover that is attached to the flexible overmold layer. The compact red light IR therapeutic element within the frustoconical shaped flange is adapted to be positioned over a portion of the breast and to apply an illuminated red light onto the portion of breast needing therapy.

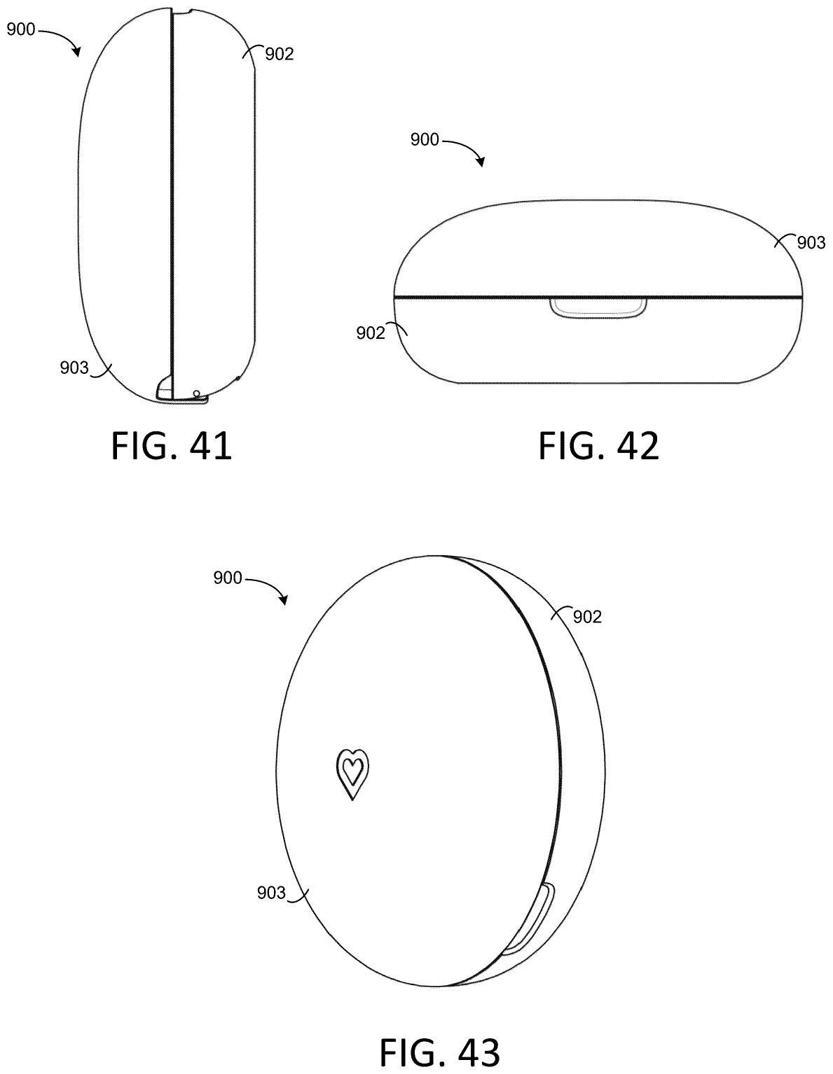

FIGS. 41-48 illustrate another exemplary embodiment of a charging case 900 adapted to receive and recharge the batteries 76 for a compact red light IR therapeutic element such as the IR nipple shield 10. FIG. 41 shows a side view of the charging case 900, FIG. 42 shows a front view of the charging case 900, and FIG. 43 shows a perspective view of the charging case 900. The charging case 900 is a compact case, being smaller in size relative to the charging case 200 illustrated in FIGS. 15-18. As shown in FIGS. 41-48, the charging case 900 is adapted to secure the IR nipple shield 10 within the housing 902 of the charging case 900.

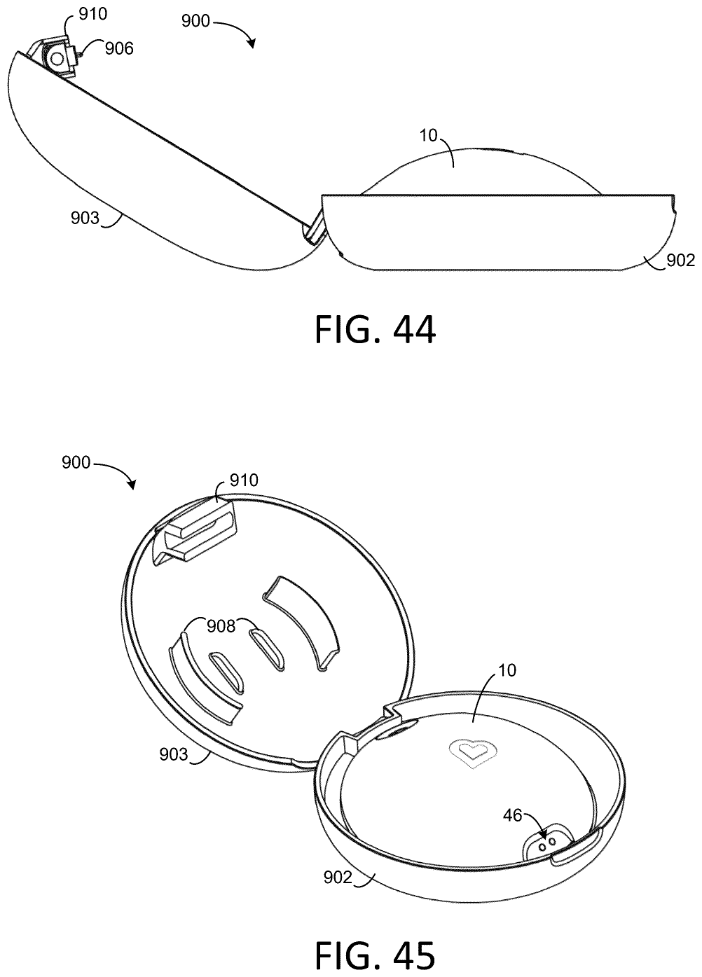

The charging case 900 includes a lid 903 attached to the housing 902 of the charging case 900. The lid 903 may be operable in a closed position (e.g., as illustrated in FIGS. 41-43) where the lid 903 is aligned over a cavity 904 fully enclosing the IR nipple shield 10 within the housing 902. The lid 903 may be positioned in an open position (e.g., as illustrated in FIGS. 44-48) where the lid 903 is displaced and/or pivoted away from the housing 902 and the cavity 904 such that a user can remove the IR nipple shield 10 from the cavity 904 or replace the IR nipple shield 10 within the cavity 904. The lid 903 can be pivotably attached to the housing 902 and can include a magnetic or mechanical closing system.

FIG. 44 shows a side view of the charging case 900 in an open position, and FIG. 45 shows a perspective view of the charging case 900 in an open position. Both FIGS. 44 and 45 also illustrate a position of the IR nipple shield 10 seated within the housing 902. As discussed above, the IR nipple shield 10 includes a pair of battery charging electrode contacts 46 which are adapted to come into contact with a pair of electrode contacts 906 (e.g., similar to mating contact 206 illustrated in FIG. 18) mutually arranged to transfer a charge through the electrode contacts 46 on the IR nipple shield 10. The pair of electrode contacts 906 may be removably attached to a slot mechanism 910 of an inner surface of the lid 903, such that when the lid 903 is closed, the electrode contacts 906 become electrically coupled to the electrode contacts 46 to facilitate charging of the IR nipple shield 10 batteries 76. Accordingly, the electrode contacts 906 of the charging case may be removably attached to the lid 903 via the slot mechanism 910.

The lid 903 may also include one or more alignment structures 908 configured to align a position of the IR nipple shield 10 within the charging case and/or restrict movement of the IR nipple shield 10 within the charging case 900. As such, even in a mobile environment (e.g., in a purse or bag) the IR nipple shield 10 is prevented from moving within the charging case 900 to prevent interruption of battery charging. In some examples, one or more mating retention magnets (no shown) may be placed within the charging case 900 to aid in proper alignment of the IR nipple shield 10 within the housing 902 so that the electrode contacts 46 on the IR nipple shield 10 are properly aligned with the electrode contacts 906 on the charging case 200 when the lid 903 is closed.

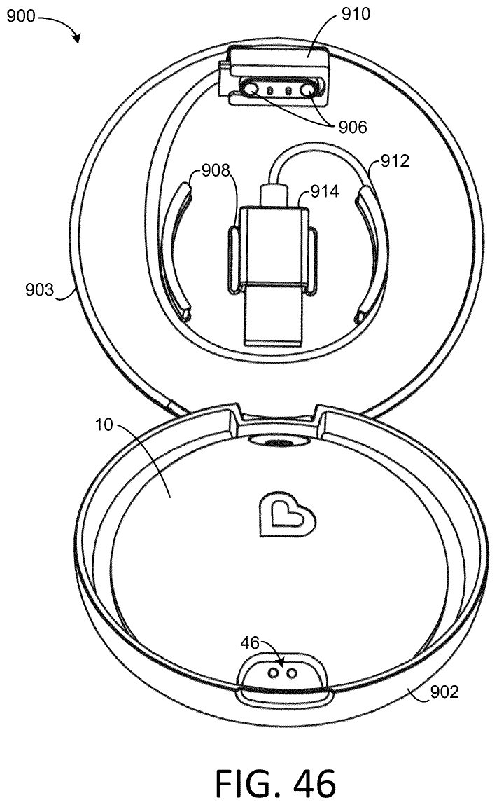

FIG. 46 illustrates a perspective view of an open charging case 900 showing an example of how the one or more alignment structures 908 may also be used to store a charging cable 912 (e.g., universal serial bus (USB) or any other suitable format associated with an electronic charging cable). Here, the charging cable 912 is configured to couple the electrode contact with a USB plug 914 via a cable. This arrangement facilitates secure storage of the charging cable 912 within the charging case 900.

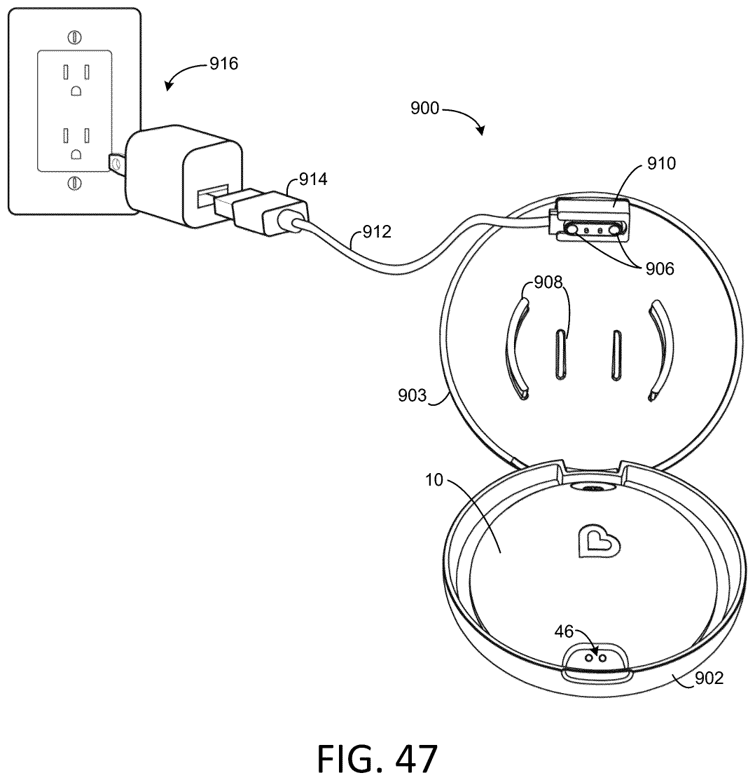

FIG. 47 illustrates the perspective view shown in FIG. 46 with an example technique for charging the IR nipple shield 10 via the charging case 910. As illustrated, the charging cable 912 may pass a current from a power source 916 (e.g., a wall outlet and an alternating current (AC) to USB adapter). When the charging case 900 is closed, the batteries 76 may be charged by a current that passes from the electrode contacts 906 on the lid 903 to the electrode contacts 46 on the IR nipple shield 10.

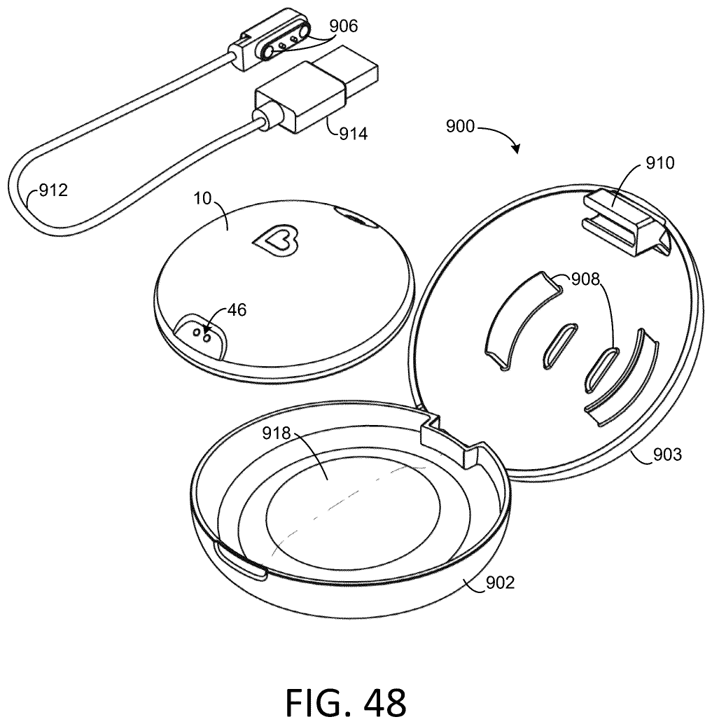

FIG. 48 is an exploded perspective view of the charging case 900. In certain aspects, an internal surface of a base of the housing may be defined by a convex or bowed-out shape 918 configured to receive the overmold layer 90 of the IR nipple shield 10. In some example, the bowed-out shape 918 may be configured to hold the IR nipple shield 10 in place within the housing 902 so that the IR nipple shield 10 does not move and charging is not interrupted by motion of the charging case 900. Although not illustrated, in some examples, the bowed-out shape 918 may include one or more mechanical slot-like fixtures configured to receive a corresponding fixture of the overmold layer 90 to prevent rotational movement of the IR nipple shield 10 when it is seated in the housing 902.

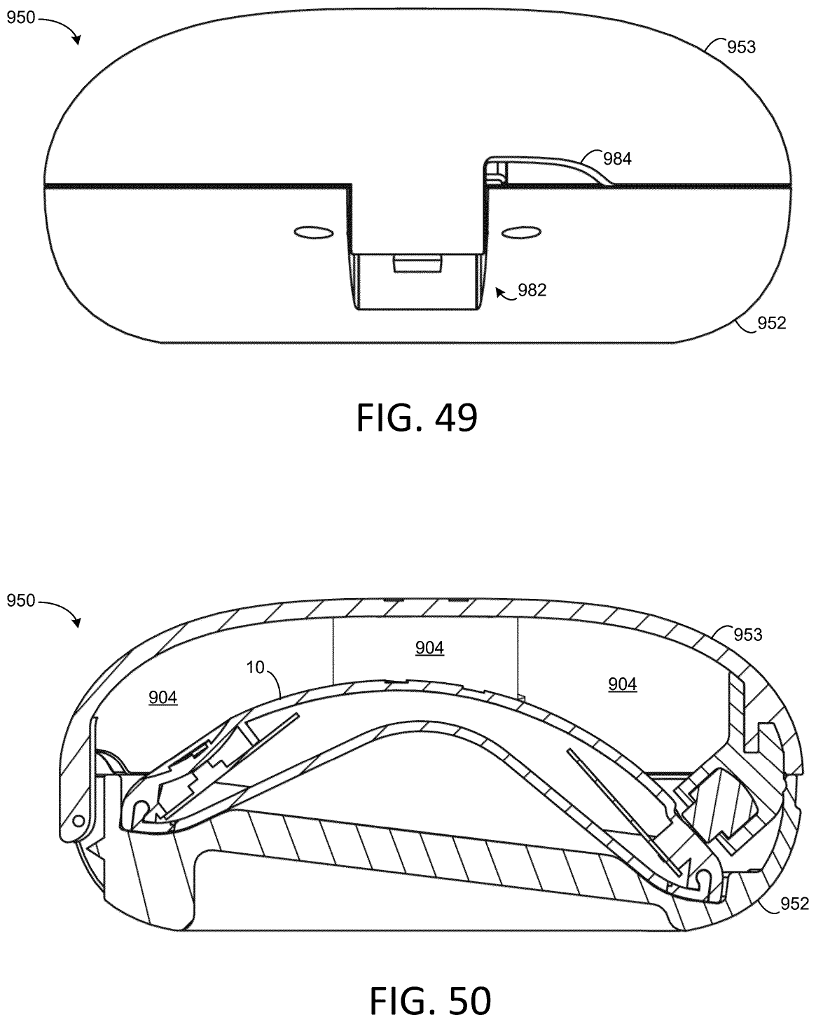

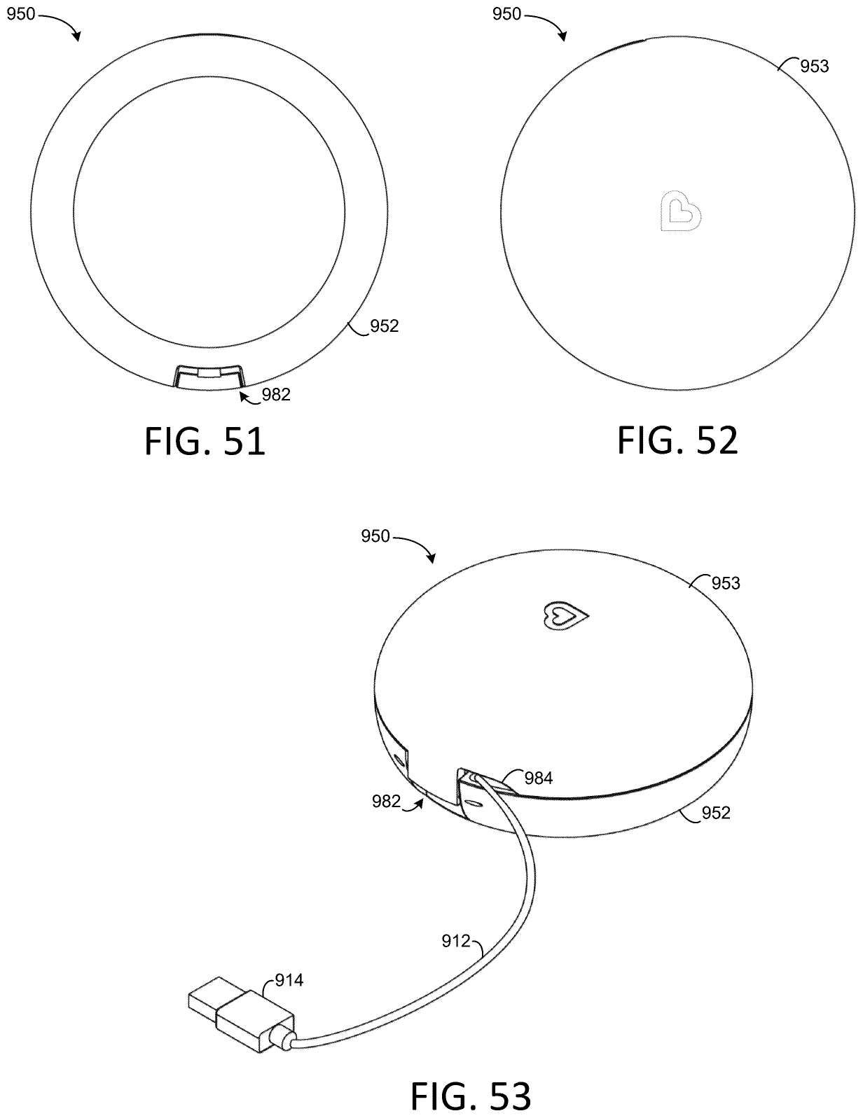

FIGS. 49-53 illustrate another exemplary embodiment of a charging case 950 adapted to receive and recharge the batteries 76 for a compact red light IR therapeutic element such as the IR nipple shield 10. The charging case 950 of FIGS. 49-53 may be similar to the charging case 900 of FIGS. 41-48 in some respects. For example, the charging case 950 may be compact relative to the relative to the charging case 200 illustrated in FIGS. 15-18. Moreover, the charging case 950 may include a housing 952, a lid 953, and a latching mechanism 982 configured to keep the charging case 950 in a closed position.

However, charging case 950 of FIGS. 49-53 may include additional features to enhance charging of the IR nipple shield 10. In one example, the lid 953 may include a cutout portion 984 configured to allow the user to pass the charging cable 912 through the charging case 950 when the lid 953 is closed.

The illustrations and examples provided herein are for explanatory purposes and are not intended to limit the scope of the appended claims. It will be recognized by those skilled in the art that changes, or modifications may be made to the above-described embodiments without departing from the broad inventive concepts of the invention. It is understood therefore that the invention is not limited to the particular embodiments which are described but is intended to cover all modifications and changes within the scope and spirit of the subject disclosure.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed:

1. A compact infrared (IR) nipple shield system, comprising:
a nipple shield device, comprising:
a cover;
an IR light-emitting diode (LED);
a flexible circuit board electrically connected to the IR LED; and
a flexible overmold layer connected to the cover, wherein the flexible circuit board and the IR LED are disposed between the cover and the flexible overmold layer, and wherein the cover is adapted to be secured over a woman's nipple and cause the IR LED to illuminate IR light onto the woman's nipple.

2. The compact IR nipple shield system of claim 1, further comprising a charger associated with the nipple shield device, wherein the charger is configured to wirelessly charge the nipple shield device.

3. The compact IR nipple shield system of claim 1, further comprising a charger associated with the nipple shield device, wherein the charger comprises:
a base having a cavity adapted to receive a portion of the nipple shield device;
a power source adapted to recharge a battery source disposed within the nipple shield device; and
a circuit board adapted to provide various routines to recharge the nipple shield device.

4. The compact IR nipple shield system of claim 3, wherein:
the nipple shield device further comprises a first magnetic material disposed onto the flexible overmold layer;
the charger further comprises a second magnetic material disposed onto the base; and
the second magnetic material is configured to receive the first magnetic material.

5. The compact IR nipple shield system of claim 3, wherein the charger further comprises:
a lid comprising a cutout portion; and
a hinge coupled to the lid and the base, wherein the hinge is configured to cause a surface of the lid to close flush against a surface of the base, and wherein the cutout portion is configured to provide access to the cavity while the surface of the lid is closed flush against the surface of the base.

6. The compact IR nipple shield system of claim 3, wherein the charger further comprises:
a lid, and wherein the power source is removably attached to an inner surface of the lid.

7. The compact IR nipple shield system of claim 1, wherein the nipple shield device is further adapted to be positioned between a garment worn by the woman and the woman's nipple.

8. The compact IR nipple shield system of claim 1, wherein the cover and the flexible overmold layer form a reservoir configured to receive and collect breast milk.

9. The compact IR nipple shield system of claim 1, further comprising a charger associated with the nipple shield device, wherein the nipple shield device further comprises a wireless power receiving coil configured to receive inductive power, and wherein the charger further comprises a wireless power transmitting coil configured to transmit the inductive power.

10. The compact IR nipple shield system of claim 1, wherein the nipple shield device further comprises:
one or more processors; and
a wireless transceiver configured to communicate wireless signals between the one or more processors and an external device.

11. The compact IR nipple shield system of claim 1, further comprising a charger associated with the nipple shield device, wherein the charger further comprises:
one or more processors; and
a wireless transceiver configured to communicate wireless signals between the one or more processors and an external device.

12. A compact infrared (IR) nipple shield device, comprising:
a cover;
an IR light-emitting diode (LED);
a flexible circuit board electrically connected to the IR LED; and
a flexible overmold layer connected to the cover, wherein the flexible circuit board and the IR LED are disposed between the cover and the flexible overmold layer, and wherein the cover is adapted to be secured over a woman's nipple and cause the IR LED to illuminate IR light onto the woman's nipple.

13. The compact IR nipple shield device of claim 12, further comprising:
one or more processors; and
a wireless transceiver configured to communicate wireless signals between the one or more processors and an external device.

14. The compact IR nipple shield device of claim 12, further comprising:
a wireless power receiving coil configured to receive inductive power from a wireless power source.

15. The compact IR nipple shield device of claim 12, further comprising a battery, wherein the flexible overmold layer comprises a first magnetic material disposed onto the flexible overmold layer, wherein the first magnetic material is configured to receive a second magnetic material during charging of the battery.

16. The compact IR nipple shield device of claim 12, further comprising a breast pump connector removably attached to the cover, wherein the cover comprises a cutout configured to allow breast milk to pass through the cover and into the breast pump connector.

17. The compact IR nipple shield device of claim 16, wherein the breast pump connector comprises a suction cup, and wherein the breast pump connector is configured to be manually operated.

18. A method of applying therapeutic infrared (IR) light to injured skin, comprising:
securing, over a woman's nipple, a conically-shaped nipple shield device comprising:
a cover adapted to be secured over the woman's nipple,
an IR light-emitting diode (LED),
a flexible circuit board electrically connected to the IR LED, and
a flexible overmold layer connected to the cover wherein the flexible circuit board and the IR LED are disposed between the cover and the flexible overmold layer; and
illuminating the woman's nipple with IR light from the IR LED.

19. The method of claim 18, wherein the conically-shaped nipple shield device is adapted to be positioned between a garment worn by the woman and the woman's nipple.

20. The method of claim 18, wherein the cover and the flexible overmold layer form a reservoir configured to receive and collect breast milk.
